(19) United States
(12) Patent Application Publication
(10) Pub. No.: US 2013/0226114 A1
(43) Pub. Date: Aug. 29, 2013

(54) DRAINAGE SYSTEM

(71) Applicants: Shayna Massi, Chicago, IL (US); Anthony M. Looper, Lake Zurich, IL (US); John A. Krueger, Muskego, WI (US); James Kantola, Waukegan, IL (US); Kelly Landsman, Milwaukee, WI (US)

(72) Inventors: Shayna Massi, Chicago, IL (US); Anthony M. Looper, Lake Zurich, IL (US); John A. Krueger, Muskego, WI (US); James Kantola, Waukegan, IL (US); Kelly Landsman, Milwaukee, WI (US)

(21) Appl. No.: 13/408,572

(22) Filed: Feb. 29, 2012

(51) Int. Cl. A61M 1/00; A61M 27/00

(52) U.S. Cl. 604/318; 604/327; 604/319

(57) ABSTRACT

A medical drainage system may include a pair of inlet tubes separated by a pump bulb and a valve chamber, where the valve chamber is in fluid communication with a first inlet tube across a one way valve. A second one-way valve may be provided in the entrance to a drainage container at an inlet tube end opposite the pump bulb. The drainage container may be rigid or flexible and may be equipped with emptying means such as a drain valve, connector for suction source, or other emptying/draining means that can be used to partially or wholly void the container contents (e.g., for sampling, testing, or disposal).

### TECHNICAL FIELD

Embodiments disclosed herein generally relate to a medical drainage system. More particularly, disclosed embodiments relate to devices for draining and containing ascites or other fluid from a patient's body region.

### BACKGROUND

Fluid accumulation in a mammalian body may occur due to trauma (including surgical trauma or other injury) in body regions not designed to accommodate and relieve such accumulation. One particular area prone to abnormal fluid accumulation is the region between sheets of tissue covering the outside of the lung and lining the chest cavity, known as the pleural space. Although a healthy, functioning pleural space contains approximately 5-20 mL of fluid. The pH, glucose and electrolytes of the fluid are equilibrated with plasma, but the fluid is relatively protein-free. The fluid is the result of the hydrostatic-oncotic pressure of the capillaries of the parietal pleura. About 80-90% of the fluid is reabsorbed by the pulmonary venous capillaries of the visceral pleura, and the remaining 10-20% is reabsorbed by the pleural lymphatic system. The turnover of fluid in the pleural space is normally quite rapid—roughly 35 to 75% per hour, so that 5 to 10 liters of fluid move through the pleural space each day. As a result, any disruption in fluid turnover/release may result in over-accumulation of fluid in the pleural space, known as pleural effusion. Complications associated with pleural effusion may include dyspnea, tachycardia, cough, breathing difficulty, and chest pain because the lungs are prevented from fully expanding upon breathing. Pleural effusion may occur as a secondary condition related to—for example—trauma, cancer, nephrotic syndrome, kidney disease, pancreatitis, congestive heart failure, or cirrhosis. Increased mortality risk often occurs with pleural effusion.

Although pleural effusion typically occurs toward the end of terminal malignancies such as breast cancer, it occurs earlier in other diseases. Therefore relieving the clinical manifestations of pleural effusion is of a real and extended advantage to the patient. For example, non-breast cancer patients with pleural effusion have been known to survive for years. Pleural effusion and/or other unwanted fluid accumulation conditions may be treated in a variety of manners, often depending upon other patient indications. Fluid drainage procedures, such as thoracentesis, may be used to provide patient relief. Thoracentesis includes introduction of a drainage catheter into the pleural space through an incision in the chest cavity, after which fluid may be removed using—for example—a syringe or a vacuum source. Pleurodesis is another treatment procedure, where fluid is prevented from accumulating by effecting sealing of space between pleura with a sclerotic or other appropriate material, often after first draining existing fluid. Another method to treat pleural effusion is to surgically implant a chest tube or catheter such that fluid accumulation can constantly or periodically be removed without invasive surgery. The implanted catheter may be connected to an external catheter or drainage tube by a one-way valve mechanism, which permits fluid drainage through the use of a negative pressure source, such as a vacuum. Examples of such a system include those described below in this background section.

Excess fluid that may accumulate in the peritoneal cavity (the space between the tissues lining the abdomen and abdominal organs) is known as ascites. Disorders associated with ascites may include cirrhosis and related ailments, clots in the veins of the liver (e.g., portal vein thrombosis), colon cancer, congestive heart failure, constrictive pericarditis, hepatitis, infections (e.g., tuberculosis), liver cancer, nephrotic syndrome, ovarian and/or endometrial cancers, pancreatitis, pancreatic cancer, and others. Treatment may include paracentesis or other methods.

Both excess fluid accumulation conditions may be treated with a drainage apparatus of the type shown in FIG. 1. The apparatus 100 is shown as installed in a patient body and includes a drainage container 114. The drainage container 114 is removably attached by a proximal tube 110 at a valve 60 to a distal catheter 12. The valve 60 may be configured in any number of ways known in the art for attaching catheters together in a fluid-patent manner (which may include a two-part valve), including for example those structures described in U.S. Pat. Publ. 2012/0004644 to Strole, et al., which is incorporated herein by reference. The proximal portion attached to the distal catheter 12 may be configured to be self-sealing when disconnected from the proximal tube 110. The proximal end portion of the distal catheter 12 is shown indwelling the patient, disposed through the body wall 21 into an intra-body space 23, which may be—for example—a pleural, peritoneal, or other body lumen. That proximal portion includes a sealing cuff 19 and a flexible fluid-intake length 14 including apertures 18, shown in the intra-body space 23. This structure may be better understood with reference to U.S. Pat. No. 5,484,401, which is incorporated herein by reference, and with reference to commercial products marketed under the name PleurX by CareFusion® of San Diego, Calif.

It may be desirable to provide a drainage system configured for use by patients and/or by caregivers where that system is easy to use and safely contains the medical waste it accrues during use.

### BRIEF SUMMARY

In one aspect, embodiments disclosed herein may include various medical drainage systems, as well as methods for making, assembling, and or using said systems.

In one aspect, embodiments disclosed herein may include a first inlet tube configured to be near to and in fluid communication with a patient body; a first one-way valve adjacent and controlling fluid communication with the first inlet tube; a valve chamber adjacent the first one-way valve and opposite the first inlet tube, where the valve chamber includes a lumen in controlled fluid communication with the first inlet tube across the first one-way valve, and where fluid flow through an interior portion of the valve chamber lumen is at least partially externally observable through a wall of the valve chamber; a drainage container including an evacuation port; a second inlet tube disposed in fluid communication with the valve chamber, between the valve chamber and the drainage container; a pump bulb including a pump bulb lumen disposed adjacent the second inlet tube in fluid communication with the valve chamber and with the first inlet tube, where the pump bulb is located between the valve chamber and the drainage container; and a second one-way valve disposed between the second inlet tube and the drainage container.

In certain embodiments, the presence and operation of a pump bulb with one or more filters proximal and/or distal thereof may provide effective drainage for a patient. In certain embodiments, the use of transparent and/or translucent materials in a filter chamber, tubing, and/or drainage container may provide for patient assurance of proper/continuing drainage and may provide clinical value for an observer of device contents. The disclosed system may also provide for ease of hygienic use under a variety of circumstances. Certain embodiments may provide a length of larger inner diameter drainage tubing along at least one portion.

### BRIEF DESCRIPTION OF THE DRAWINGS

FIG. 1 shows a prior art drainage system;

FIG. 2 shows one embodiment of a medical drainage system;

FIGS. 3A-3C show one valve embodiment useful in a container of a medical drainage system;

FIGS. 4A-4C show a volume indicia for a “lie-flat” bag-type container.

### DETAILED DESCRIPTION

In the present application, the term “proximal” is generally used to refer to an end or portion of the system or device nearer the patient, while the term “distal” refers to the opposite end or portion.

Embodiments are described with reference to the drawings in which like elements generally are referred to by like numerals. The relationship and functioning of the various elements of the embodiments may better be understood by reference to the following detailed description. However, embodiments are not limited to those illustrated in the drawings. It should be understood that the drawings are not necessarily to scale, and in certain instances details may have been omitted that are not necessary for an understanding of embodiments disclosed herein, such as—for example—conventional fabrication and assembly.

### GENERIC DESCRIPTION

Various embodiments will be described more fully hereinafter. The invention is defined by the claims, may be embodied in many different forms, and should not be construed as limited to the embodiments set forth herein; rather, these embodiments are provided so that this disclosure will be thorough and complete, and will fully convey enabling disclosure to those skilled in the art. As used in this specification and the claims, the singular forms “a,” “an,” and “the” include plural referents unless the context clearly dictates otherwise.

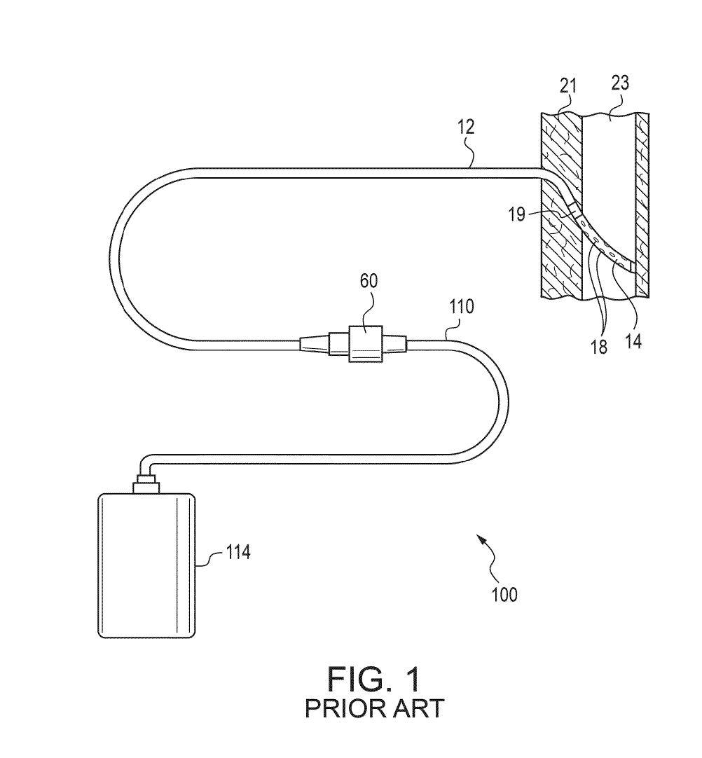

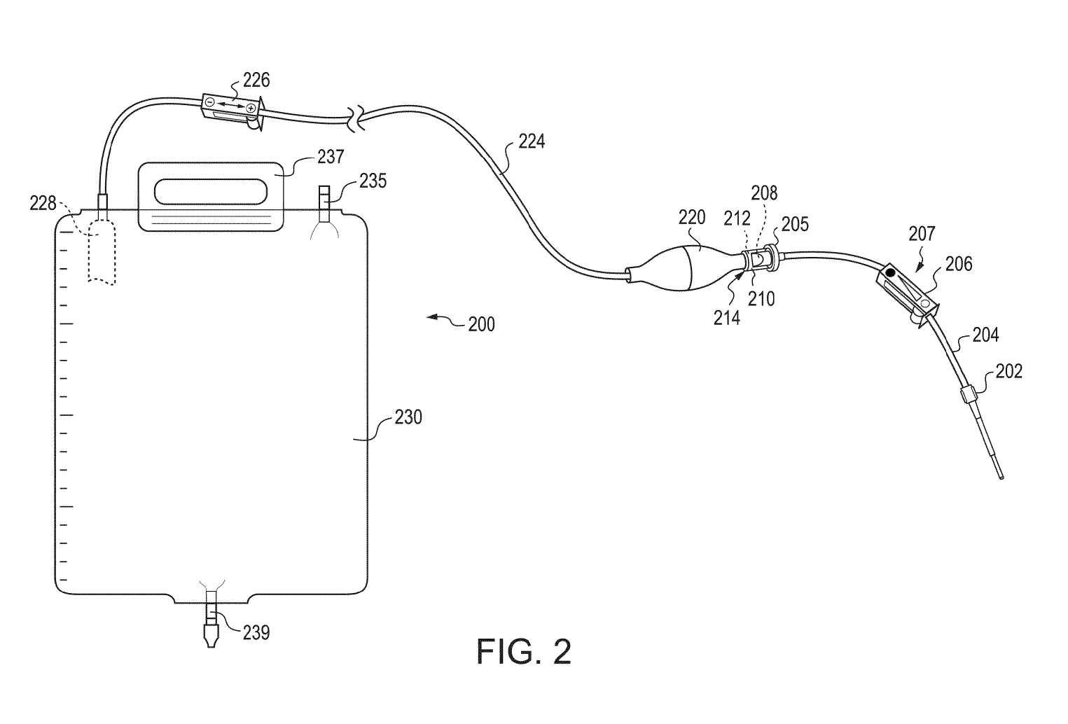

One embodiment of a medical drainage system 200 is described with reference to FIG. 2. The system 200 is shown with a proximal end connector 202 configured to engage with, for example, an indwelling pleural or peritoneal drainage catheter of the type shown in FIG. 1. In some embodiments, the system 200 may include a connection with a peritoneal drainage catheter or a pleural drainage catheter (e.g., as shown in FIG. 1, or having another configuration). The connector 202 is attached to a first inlet tube 204, which is configured to provide a path of fluid communication from the patient toward a drainage container 230. A first roller clamp 206 may be provided on the first inlet tube 204. The roller clamp 206 may be used to control (and stop) rate of flow through the first inlet tube 204. In this manner, a user may control the flow/drainage rate with an easily operable mechanism that does not require moving the container 230.

The first roller clamp 206 configured to adjustably modulate a fluid flow through the portion of inlet tube 204 that it engages. The roller clamp 206 may include visual and/or tactile indicia 207 showing how to adjust the roller clamp for increased flow, decreased flow, and/or no flow. Roller clamps are known in the art to have a variety of constructions that may be used within the scope of the present disclosure. A filter chamber 205 may be provided proximal or distal of the first roller clamp 206 between the connector 202 and a first one-way valve. The filter chamber may be configured to capture tissue without substantially impeding fluid flow through the filter chamber toward the first one-way valve.

A valve chamber structure 210 is attached to the first inlet tube 204 by the first one-way valve 208, which controls fluid communication between the first inlet tube 204 and the valve chamber 210 (limiting that communication to flow from the tube into the valve chamber, but not in reverse). Stated differently, the valve chamber is in controlled fluid communication with the first inlet tube across the first one-way valve. The first one-way valve 208 may be configured as a large-bore duckbill valve or other check-valve structure (that preferably will not have its function impaired by passage therethrough of the bodily drainage materials contemplated herein). The valve chamber 210 includes an interior lumen 212 defined by the valve chamber wall 214. In certain preferred embodiments, fluid flow through an interior portion of the valve chamber lumen 212 is at least partially externally observable through a wall of the valve chamber. In some embodiments, at least a portion of the valve chamber wall 214 is transparent or at least translucent so that an interior portion of the valve chamber lumen 212 is viewable therethrough. In those or other embodiments, the valve chamber 210 may include a flow indicator (not shown) configured to indicate when fluid is flowing through the chamber 210. Fluid flow indicators are well known in the art and may be provided in a manner to indicate the presence/absence of flow and/or to indicate rate and/or volume of flow.

A pump bulb 220 is disposed distal of the first inlet tube 204 and the valve chamber 210. The pump bulb 220 may be immediately adjacent the valve chamber 210, or it may be separated by an intervening tube length. The pump bulb 220 is adjacent a second inlet tube 224, which is disposed opposite the valve chamber 210. The construction and operation of a pump bulb 220 is well-known in the art. It may be constructed of a flexible material (e.g., silicon), and its operation—in conjunction with the first one-way valve 208 (and downstream second one-way valve 228) will pull a vacuum in the first inlet tube 204. The second inlet tube 224 may have a thicker wall and/or a larger inner diameter than the first inlet tube 204.

The tubing outer diameter and wall thickness for the first inlet tube 204 and the second inlet tube 224 preferably are different. In one preferred embodiment, the first inlet tube 204 includes a smaller outer diameter and/or wall thickness as compared to the second inlet tube 224. In one aspect, this will allow a patient more easily to actuate the first roller clamp 206. In particular, many patients who will use the device embodiments described herein suffer from limited strength and/or dexterity (due, e.g., to one or more advancing age, debilitation by illness, side effects of radiation and/or chemotherapy, etc.). As such, it will be advantageous to provide a system easily operable by persons having these or other physical limitations. In one embodiment, the first inlet tube 204 may include an inner diameter of about 0.113 inches inner diameter, about 0.187 inches outer diameter, with a wall thickness of about 0.037 inches and Shore A 70 durometer.

In those and/or other embodiments, it may also be preferable to include a larger, less pliable second inlet tube 224. Many patients may allow the container 230 to rest on the floor or another surface during use. Providing a larger, less pliable second inlet tube decreases the likelihood that it will become kinked, pinched, or otherwise distorted in a manner that would impede drainage. In one embodiment, the second inlet tube 224 may include an inner diameter of about 0.188 inches inner diameter, about 0.313 inches outer diameter, with a wall thickness of about 0.0625 inches and Shore A 78 durometer.

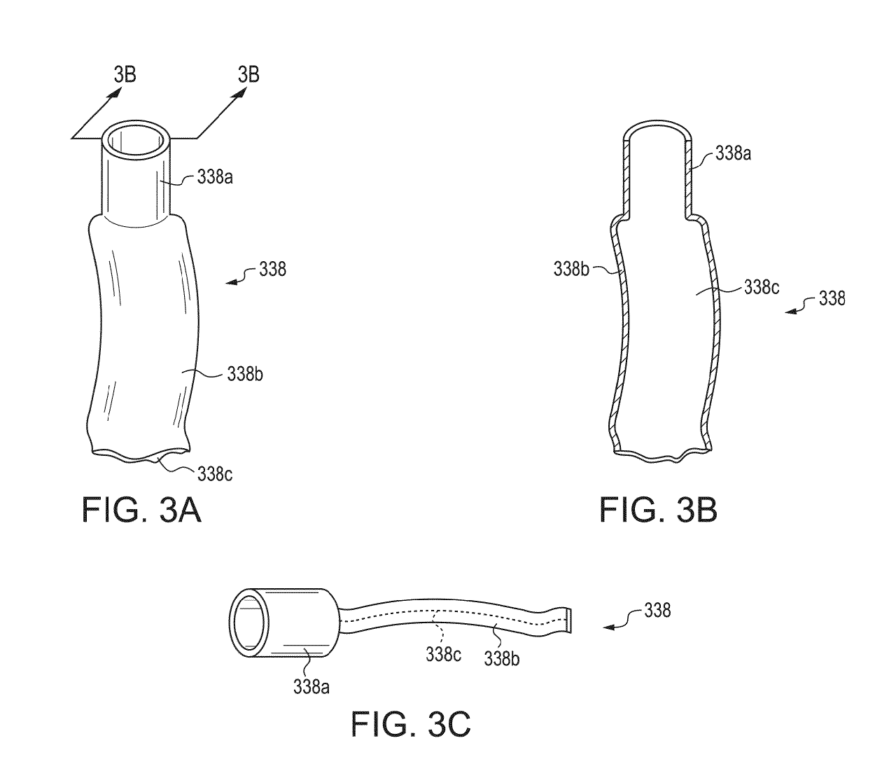

The second inlet tube 224 is attached to and disposed in fluid communication with the drainage container 230 via a second one-way valve 238. The second one-way valve 238 (and/or the first valve 208) may be configured as, for example, a flutter valve. An example of a flutter valve 338 contemplated is shown in FIGS. 3A-3C as a modified duckbill-type valve. FIG. 3A shows a front view of the valve 338, which includes a semi-flexible or rigid neck ring portion 338a attached to a very flexible flap portion 338b. FIG. 3C shows a side external view of the flutter valve 338 As shown in the longitudinal section view of FIG. 3B, taken along line 3B-3B of FIG. 3A, the edges of the flap portion 338b are attached and form an openable lumen 338c therebetween, which is open through the neck portion 338a. The default configuration of the flap portion 338b is to keep the lumen 338c closed by remaining pressed together (e.g. by material bias, by surface attraction via van der Waals forces, and/or when there is even a slight vacuum from the neck end). The lumen 338c opens to permit flow therethrough when fluid pressure is present from the neck end through the lumen 338c.

A second roller clamp 226 may be disposed around the second inlet tube 224, and may include structure and function like that described above for the first roller clamp 206. A portion, up to the entirety, of the second inlet tube 224 may be transparent or translucent, which may permit visualization of material passing therethrough. The color, appearance, and/or flow rate of that material may provide clinical value.

The drainage container 230 may be configured as a bag, box, or bottle. It may be constructed of flexible, semi-flexible, or rigid materials, and it may include one or more feature(s) that permit/facilitate its being mounted (e.g., hung from) some ancillary structure. A filter vent 235 may be provided on the drainage container 230, where the filter vent 235 is configured to allow gas to evacuate the drainage container as it fills with fluid. The filter vent 235 may include a filter material such as activated charcoal or another material configured to control the release of odors and/or airborne particles (and, it may also include passive or active antimicrobial agents). The container 230 preferably includes an evacuation port structure or assembly 239 configured to allow the contents of the container to be removed. The materials of which the container 230 is constructed may vary: it may be generally rigid (e.g., configured to resist significant shape change when subjected to a vacuum of less than 120 mmHg), or it may include a generally flexible polymer construction configured to conform generally to an internal volume of material in bag-like fashion.

The drainage container 230 may be graduated with markings that will allow an observer to view the volume (e.g., in mL) of material in the container, and one or more surfaces of the container 230 may be transparent or translucent to facilitate such observation. A mounting and/or carrying structure such as a loop 237 may be provided that will enable a user to hang/suspend the container from—for example—a bed-mounted hook or a wheelchair accessory hook, or to more easily manually carry the container 230. The handle will also aid the user in holding the drainage container when emptying it after drainage.

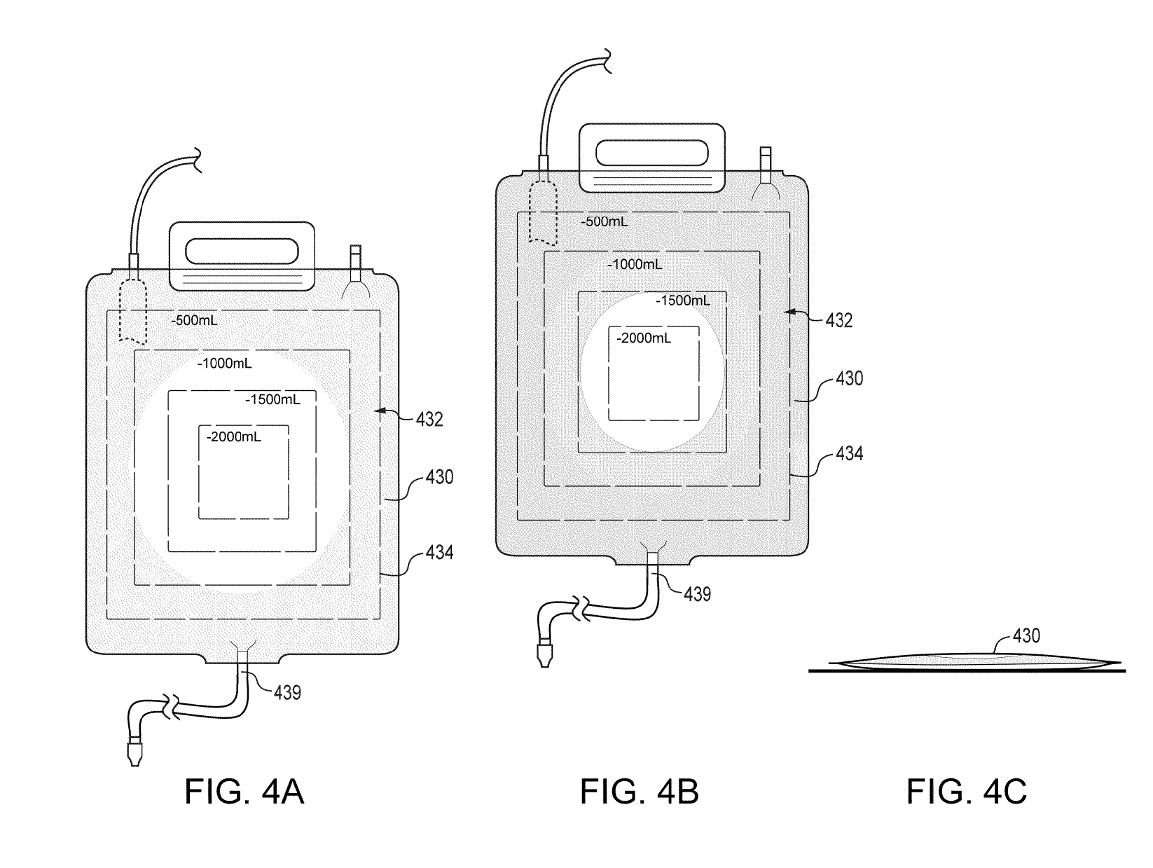

In some embodiments, the drainage container, embodied as a bag with flexible walls, may be constructed to rest on a flat surface rather than being suspended (e.g., a “lie-flat” configuration). FIGS. 4A-4C, each rendered diagrammatically for purposes of simplified illustration, show one such embodiment as a drainage container 430. The drainage container 430 may include a transparent or translucent upper-facing wall 432 configured to be oriented upward opposite a side wall resting on a generally horizontal surface that preferably is below the patient and/or below the first inlet tube as described above (e.g., on a floor, low table, bed surface). A set of graduation indicia 434 may be provided on the upper-facing wall 432 that will allow a patient or other person to assess a visual estimate of the bag's volume. Specifically, without being quantitatively specific, the graduation indicia 434 may provide for visual estimation of the bag's contents of volume of material and/or remaining space by visualization of the margin of an air bubble through the upper wall 432, as shown by shading of simulated bag contents in a sample 2 L bag. FIG. 4A shows the container 430 as containing a lower volume of drained material. FIG. 4B shows the container 430 as containing a greater volume of drained material, and FIG. 4C shows a side view of the container 430 of FIG. 4B.

The evacuation port preferably is disposed at an end region of the container 230 opposite of the second one-way valve 238, which serves as an ingress for fluid from the tube 224. The evacuation port 239 may be constructed in a variety of manners, depending upon a desired implementation. For example, the evacuation port 239 may be constructed with a connector configured to interface with and provide fluid communication to a standard medical wall suction fixture. It may include a twist-open drain spout of any type known in the art for fluid drainage. It may include a tube with a removable clamp, or some other simple drainage control mechanism.

In some embodiments, it may be preferable to include an evacuation means in addition to or instead of the port 239. Such embodiments may facilitate a patient doing self-treatment/drainage or another caregiver (e.g., whether in or out of a hospital) the opportunity to dispose of the captured material (e.g., into a toilet to be flushed). The following potential embodiments will readily be understood by those of skill in the art without need for pictorial illustration. For example, a tear-off portion of the container 230 may be provided (e.g., on an upper corner including a filter vent 235). Instead of the port 239, a lower-end elongate drain tube may be provided that is sealed with a frangible clamp or other structure that may be removed or cut off to permit drainage. This elongate tube can be advantageous because placing it into a toilet bowl or other disposal receptacle can minimize the likelihood of splashing of the ascites material, pleural fluid, or other drained material (which may be particularly important for chemotherapy patients where the drainage material may contain non-biological matter). Other options include providing a “pull-tab” style of drainage port such as the type commonly now used in beverage containers (e.g., milk or juice jugs) having a plastic spout.

Each of these and other available options for container drainage preferably will render the container unusable after a single use to decrease the likelihood of infection and/or other hygienic issues associated with attempting to re-use an ascites drainage container. The preferred drainage means also will ease patient's emptying of the container 230 while minimizing likelihood of splashback of the discarded contents. One preferred method and structure for disposal includes a drainage tube sufficiently long to have an open (whether cut-off, unclamped, or otherwise opened) end submerged in a toilet bowl, or extended at least partially down into a sink/tub drain where water can be used to flush down drained material. One such elongate drainage tube 439 is shown in FIG. 4A.

The system may be operated by actuating the pump bulb to initiate suction/drainage after connected to the patient. In certain embodiments, a pump bulb or syringe may be located elsewhere along or out-of-line from the inlet tube(s) and/or opposite the container from the inlet tubes, the inlet tube(s) may be preloaded with a saline or other solution that can be released into the container to prime the system and initiate drainage flow, and/or wall suction may be used.

In some embodiments, the system may be provided as part of a kit. In one exemplary kit, at least one (preferably a plurality of) containers 230 is provided, rolled-up for compact transport and storage in a kit that also contains at least one inlet tube assembly including first and second inlet tubes, with an intervening pump bulb and valve chamber (e.g., as shown in FIG. 2). A roller clamp may also be included. In another exemplary kit, at least one vacuum bottle may also be included to provide a greater vacuum than may be attained with the pump bulb (e.g., to provide assistance if the pump bulb and bag-type container 230 are not operating to drain at a desired rate and/or quantity. In such a kit, the distal end of the inlet tube is configured to detach from the bag-type container and attach to the vacuum bottle.

Those of skill in the art will appreciate that embodiments not expressly illustrated herein may be practiced within the scope of the claims, including that features described herein for different embodiments may be combined with each other and/or with currently-known or future-developed technologies while remaining within the scope of the claims. Although specific terms are employed herein, they are used in a generic and descriptive sense only and not for purposes of limitation. It is therefore intended that the foregoing detailed description be regarded as illustrative rather than limiting. And, it should be understood that the following claims, including all equivalents, are intended to define the spirit and scope of this invention. Furthermore, the advantages described above are not necessarily the only advantages of the invention, and it is not necessarily expected that all of the described advantages will be achieved with every embodiment.

We claim:

1. A medical drainage system configured for draining fluid from a patient body, the system comprising:
a first inlet tube configured to be near to and in fluid communication with a patient body;
a first one-way valve adjacent and controlling fluid communication with the first inlet tube;
a valve chamber adjacent the first one-way valve and opposite the first inlet tube, where the valve chamber includes a lumen in controlled fluid communication with the first inlet tube across the first one-way valve;
a drainage container including an evacuation port;
a second inlet tube disposed in fluid communication with the valve chamber, between the valve chamber and the drainage container;
a pump bulb including a pump bulb lumen disposed adjacent the second inlet tube in fluid communication with the valve chamber and with the first inlet tube, where the pump bulb is located between the valve chamber and the drainage container; and
a second one-way valve disposed between the second inlet tube and the drainage container.

2. The system of claim 1, where at least a portion of the valve chamber wall is configured as transparent, translucent, including a fluid-flow indicator, or a combination thereof, such that fluid flow through an interior portion of the valve chamber lumen is at least partially externally observable through a wall of the valve chamber.

3. The system of claim 1, where at least a portion of the second inlet tube is transparent and permits visualization of material passing therethrough.

4. The system of claim 1, where the first inlet tube further comprises a connection with a peritoneal drainage catheter or a pleural drainage catheter.

5. The system of claim 1, further comprising a first adjustable roller clamp disposed around the first inlet tube and configured to adjustably modulate a fluid flow therethrough.

6. The system of claim 5, further comprising a second roller clamp disposed around the second inlet tube between the pump bulb and the second one-way valve.

7. The system of claim 1, where the evacuation port comprises a connector configured to provide fluid communication with a standard wall-suction fixture.

8. The system of claim 1, where the first one-way valve and the second one way valve each is configured to allow fluid flow only in the direction of the drainage container.

9. The system of claim 1, where the drainage container is embodied as a bag, a box, or a bottle.

10. The system of claim 1, where the drainage container comprises a generally rigid construction configured to resist significant shape change when subjected to a vacuum of less than 120 mmHg.

11. The system of claim 1, where the drainage container comprises a generally flexible polymer construction configured to conform generally to an internal material volume.

12. The system of claim 1, where one or both of the first one-way valve and the second one-way valve is configured as a flutter valve.

13. The system of claim 1, where the first one-way valve is configured as a large-bore duckbill valve.

14. The system of claim 1, further comprising at least one roller clamp disposed on one of the inlet tubes and configured to adjustably modulate a fluid flow therethrough, where at least one roller clamp includes visual and/or tactile indicia showing how to adjust the roller clamp for increased flow, decreased flow, and/or no flow.

15. The system of claim 1, where the evacuation port is disposed at an end region of the drainage container opposite of the second one-way valve.

16. The system of claim 1, further comprising a filter valve on the drainage container, the filter valve configured to allow gas to evacuate the drainage container as it fills with fluid during an operation of the system.

17. The system of claim 1, where the container is configured as a lie-flat bag with a wall including volumetric indicia for estimating a volume of contents therein.

18. The system of claim 1, where the second inlet tube includes a larger internal diameter than the first inlet tube.

19. The system of claim 1, further comprising a filter chamber between a patient-proximal system end and the first one-way valve, where the filter chamber includes a filter configured to capture tissue without substantially impeding fluid flow through the filter chamber toward the first one-way valve.

20. A patient treatment kit comprising:
at least one system according to claim 1; and
at least one vacuum bottle configured for attachment to a distal end of the second inlet tube.
